(19) United States
(12) Patent Application Publication
(10) Pub. No.: US 2015/0119372 A1
(43) Pub. Date: Apr. 30, 2015

(54) APPLICATION OF 18-METHYL-15SS,16SS-METHYLENE-19-NOR-20-SPIROX-4-EN-3-ONE SYSTEMS IN THE TREATMENT OF MENORRHAGIA, AS WELL AS INTRAUTERINE SYSTEMS CONTAINING 18-METHYL-15SS,16SS-METHYLENE-19-NOR-20-SPIROX-4-EN-3-ONE FOR TREATING UTERINE BLEEDING DISORDERS

(71) Applicant: BAYER PHARMA AKTIENGESELLSCHAFT, Berlin (DE)

(72) Inventors: Norbert Schmees, Berlin (DE); Lars Röse, Berlin (DE); Tuula Valo, Turku (FI); Katja Prelle, Ratingen (DE); Reinhard Nubbemeyer, Berlin (DE); Henriikka Korolainen, Loimaa (FI); Harri Jukarainen, Kuusisto (FI)

(21) Appl. No.: 14/396,742

(22) PCT Filed: Apr. 19, 2013
(86) PCT No.: PCT/EP2013/058152

§ 371 Date: Oct. 23, 2014

(30) Foreign Application Priority Data
Apr. 23, 2012 (DE) 10 2012 206 653.5

(51) Int. Cl. A61K 31/58

(52) U.S. Cl. CPC A61K 31/58
USPC 514/173

(57) ABSTRACT

The present invention describes intrauterine use of 18-methyl-15β,16β-methylene-19-nor-20-spirox-4-en-3-ones of the general formula (1)

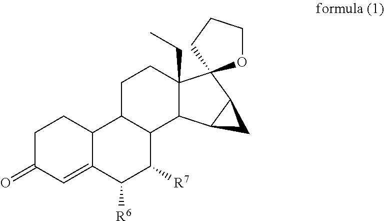

wherein R⁶ and R⁷ are hydrogen or a methylene group, in the treatment of menorrhagia, of uterine haemorrhages in general, and an intrauterine system for said use, comprising a compound of formula 1.

The present invention relates to the subject matter characterized in the patent claims, i.e. the use of 18-methyl-15β,16β-methylene-19-nor-20-spirox-4-en-3-ones in the to treatment of uterine bleeding disorders, and to an intrauterine system (IUS) for use in said indication, comprising an 18-methyl-15β,16β-methylene-19-nor-20-spirox-4-en-3-ones of the general formula I,

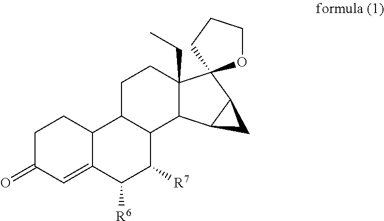

wherein R⁶ and R⁷ may be a hydrogen atom or together may be an a-methylene group.

The invention therefore relates to the use of 18-methyl-15662 ,16β-methylene-19-nor-20-spirox-4-en-3-one (compound A) or 18-methyl-6α,7α,15β,16β-dimethylene-19-nor-20-spirox-4-en-3-one (compound B)

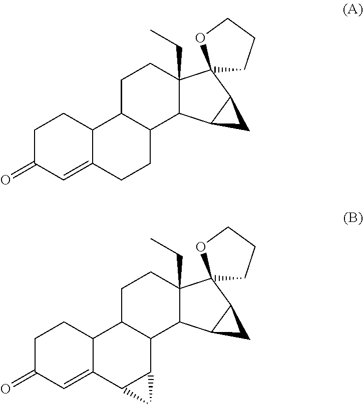

for the treatment of uterine bleeding disorders.

The invention further relates to the intrauterine use of substances (A) or (B) for the treatment of menorrhagia and to an intrauterine system for said use.

Uterine bleeding disorders mean menorrhagia, heavy menstrual bleeding (HMB) and hypermenorrhoea. Uterine bleeding disorders are known by way of different manifestations and under different names^{1,2}. These manifestations are likewise listed under uterine bleeding disorders. ¹Fraser et al. Seminars in Reproductive Medicine Vol 29, No 5 2011; 386-390² Munro et al. Fertility and Sterility_{—} Vol. 95, No. 7, June 2011; 2204-2208

Uterine bleeding disorders are also frequently caused by myomas (fibroids). The invention therefore further relates to the intrauterine use of substances (A) or (B) for the treatment of the myomas themselves and of the bleeding disorders caused by them.

Intrauterine administration of the progestins employable according to the invention may reduce or prevent any uterine haemorhage in principle. The invention therefore further relates to the intrauterine use of substances (A) or (B) for reducing or preventing uterine haemorrhages.

The progestins employable according to the invention, 18-methyl-15β,16β-methylene-19-nor-20-spirox-4-en-3-one (A) or 18-methyl-6α,7α,15β,16β-dimethylene-19-nor-20-spirox-4-en-3-one (B), and preparation thereof, are described in WO 2008000521, with the former compound (A) being disclosed there only as intermediate.

The compounds mentioned, and further substances disclosed in

WO 2008000521, are used in pharmaceutical preparations for contraception and in the therapeutical treatment of premenstrual complaints such as headaches, depressive moods, water retention and mastodynia. WO2008000521 discloses in addition to oral and transdermal dosage forms also parenteral oily injection solutions. However, WO 2008000521 does not describe an intrauterine use, the compounds being employed in an intrauterine system (IUS) nor the use of the compounds in the treatment of intrauterine bleeding disorders, more specifically menorrhagia.

Menorrhagia belongs to the menstrual disorders and denotes an excessively severe and prolonged menstrual bleeding. A blood loss of more than 80 ml per menstrual cycle is referred to as heavy menstrual bleeding.

Menorrhagias are among the most common complaints in gynaecological practice.

Possible causes are hormonal or inflammatory processes. Particularly affected by the condition are women of perimenopausal or late reproductive age. In the United States alone, over 630 000 hysterectomies are carried out each year, 12% of which are due to menorrhagia³. ³ National Inpatient Survey 2000

Anaemia and tiredness, as a result of excessive bleeding, impair the quality of life and are the cause of 12% of all gynaecological referrals.

Besides invasive treatment methods, such as the aforementioned hysterectomy or endometrial ablation which involves destroying the endometrial lining of the uterus by heat, a treatment with drugs such as naproxen, tranexamic acid, Mirena® or oral contraceptives is also suitable⁴. ⁴ Recommendations of the Royal College of Obstetricians and Gynecologists

While invasive methods can only be used on women who do not plan to have any more children or have no desire to have children, drug treatment has the advantage of not impairing fertility or, when a contraceptive is used, fertility being regained after drug taking has stopped.

A promising new form of therapy which must be mentioned is Mirena®, a levonorgestrel-containing intrauterine system (IUS) which continuously releases the active ingredient over a period of up to five years. This product is described, inter alia, in EP 0652738 B1 and EP 0652737 B1.

The action profile of Mirena® with respect to haemorrhages is based on locally induced suppression of the endometrium.

There is plenty of evidence proving that Mirena® is an extremely effective form of therapy in the treatment of menorrhagia and HMB, respectively, and superior to conventional measures⁵. A comparable effect can otherwise be achieved only by surgical methods such as endometrial ablation or resection. ⁵ Gemzell-Danielsson et al.; Acta Obstet Gynecol Scand. 2011 (11) 1177-88

Although Mirena already achieves a very high standard in the therapy of menorrhagia, the profile of Mirena® is not optimal in all cases. For instance, J. B. Dubuisson and E. Mugnier report that, in one study, about 2 in 100 women stop using

Mirena after one year because of side effects⁶. Side effects usually mentioned are transient symptoms such as mood swings, chest pain, fluid retention or skin problems (acne)⁷. ⁶ J. B. Dubuisson, E Mugnier; Contraception 2002; 66: 121-128⁷ M. Ronnerdag & V. Odlind; Acta Obstet Gynecol Scand 1999; 78: 716-721

These systemic side effects can be attributed to the comparatively high systemic stability of levonorgestrel (active ingredient in Mirena®), resulting in average plasma levels of active ingredient of around 206 pg/ml⁸. ⁸ see Fachinformation Mirena March 2011-DE9

Other undesired effects of Mirena , reported for some women, relate to cysts in the ovary⁹. ⁹ Product Monograph Mirena. 8th ed. Finland: Schering A G and Leiras Oy, August 2009

In addition, various studies show that the effect of Mirena®, in terms of reducing the amount of bleeding, has not yet reached its maximum after 2-3 months and thus a reduction in the amount of bleeding by half or to below 80 ml per menstrual cycle after said period is not yet achieved.

Thus, it may be up to 6 months until the maximum effect, i.e. a plateau, is reached with respect to the menorrhagia (HMB) indication. A comprehensive review of this has been published by Ian S. Fraser in Contraception¹⁰. ¹⁰ I. S. Fraser; Contraception 82, 2010; 396-403 (Review Article)

Further improvement regarding the above point, i.e. shortening the on-set phase by increasing the levonorgestrel (LNG) dose, is not possible because higher LNG plasma levels are expected to lead to an increase in gestagen-mediated side effects.

In summary, it can be concluded that the available drug therapies are based on induction of amenorrhoea (Mirena®), hormonal regulation (oral contraceptives), inhibition of fibrinolysis (tranexamic acid) and inhibition of inflammation (non-steroidal anti-inflammatory drugs). Besides hysterectomy and endometrial ablation, Mirena is currently the most effective therapy for HMB.

There is therefore a need for finding other gestagens useful for treating menorrhagia, that are sufficiently potent in order to be suitable for intrauterine long-term administration.

In addition, the compounds should exhibit a rapid onset, i.e. the therapeutic action should commence more quickly than with the levonorgestrel-based Mirena , even after a relatively short period of use.

In addition, the substances employed should not have any androgenic properties.

We have found that this object is achieved by using compounds of formula (I)

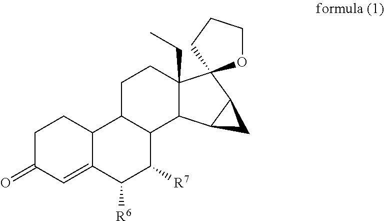

wherein R⁶ and R⁷ are a hydrogen atom or together are an a-methylene group, with preference being given to intrauterine use.

Surprisingly, when using 18-methyl-15β,16β-methylene-19-nor-20-spirox-4-en-3-one or 18-methyl-6α,7α,15β,16β-dimethylene-19-nor-20-spirox-4-en-3-one in rats, we were able to demonstrate a differentiating action between local (uterus) and systemic (peripheral tissue) effects, meaning that peripheral effects and therefore gestagen-caused side effects have been reduced.

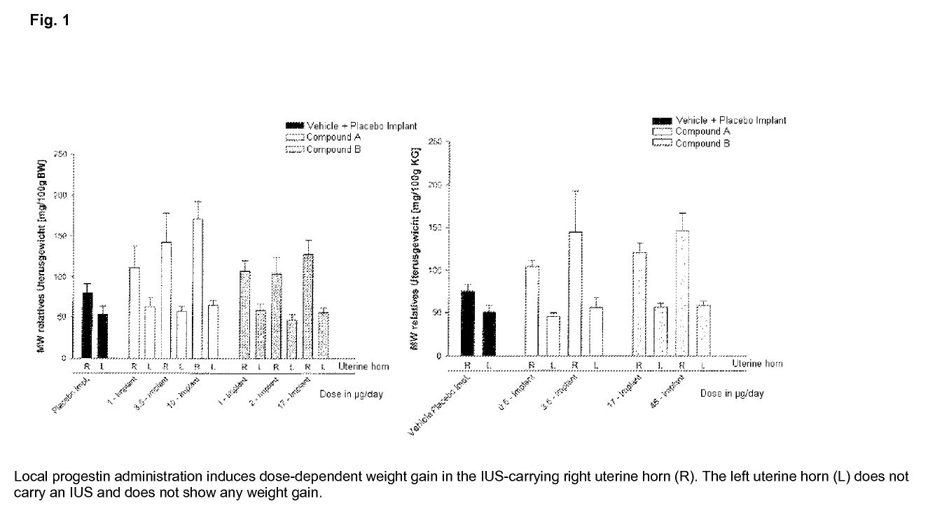

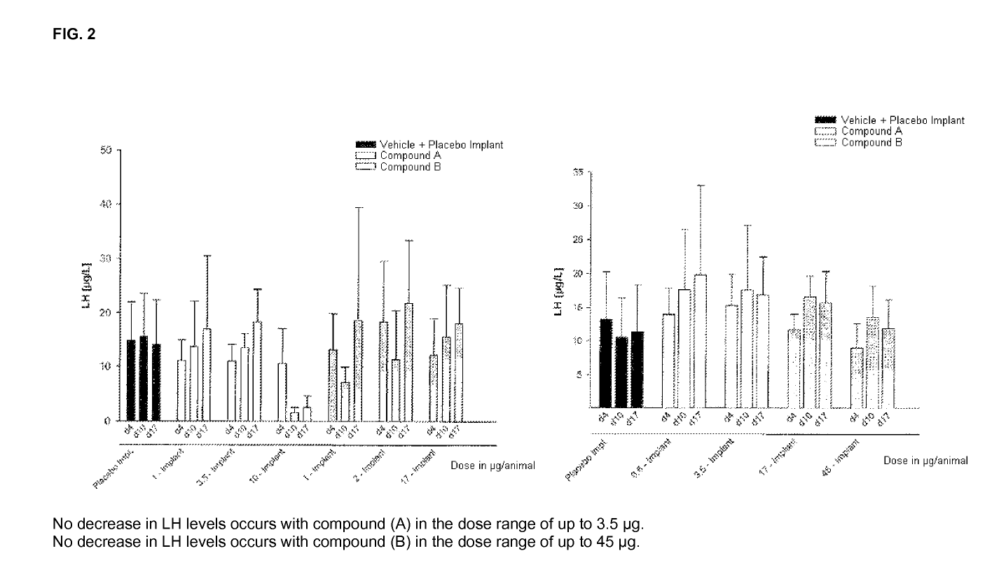

This effect was demonstrated by comparing the local effects in the uterus (weight increase, see Example 1; FIG. 1/4) and by the systemic effect such as, for example, lowering of the LH level in ovary-resected rats (FIG. 2/4).

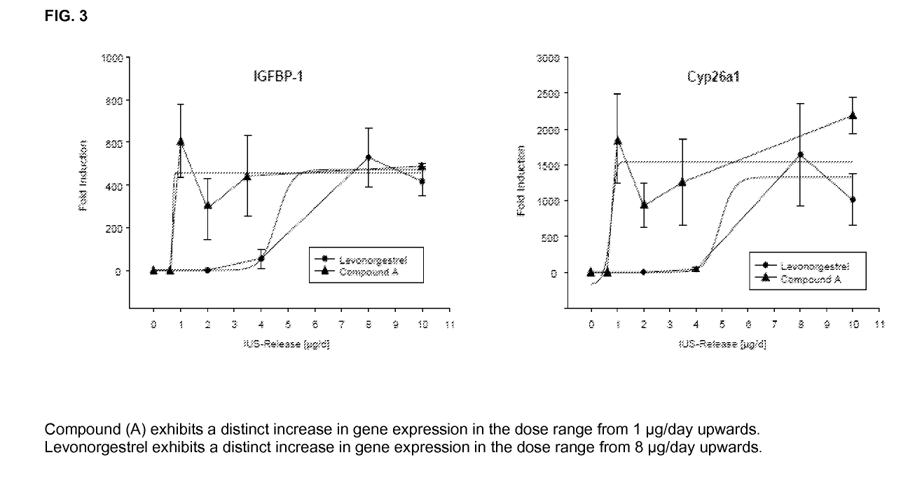

Compared with LNG, the substances also have increased local potency, as shown by the strong induction of corresponding marker genes in the gene expression experiment. Thus, the anti-oestogenic effect of gestagens on the uterus is mediated inter alia by IGFBP-1. FIG. 3/4 shows that IGFBP-1 gene expression is induced by compound A, even at a rate of release from the IUS that is approx. 7× lower than with levonorgestrel.

As furthermore demonstrated in comparative transactivation studies (see Example 2), the substances employed according to the invention have an androgenic effect that is at least 10 times lower compared to LNG. This property, still enhanced by the marked dissociation of local vs. systemic, shows that, even with local uterine uses of very high doses in comparison with levonorgestrel, no systemic androgenic effects (e.g. acne) are expected, even if systemic concentrations comparable to levonorgestrel with Mirena® uses were present.

The compounds are therefore outstandingly suited to being used in the treatment of uterine bleeding disorders such as menorrhagia. Preference is given here to an intrauterine administration by means of IUS.

An intrauterine system which may be utilized is a polymer system, as is employed, for example, with Mirena®.

A person skilled in the art is familiar with the preparation of an IUS which is carried out as described, for example in EP 0 652 738 B1.

Thus the active ingredient (A) or (B) is first made with a polymeric support material into a central rod (core). The active ingredient may be admixed with the polymeric support material, for example polydimethylsiloxane (PDMS), at any ratio.

After the shaping process, i.e. after vulcanization, the core prepared in this way is normally surrounded in a second step by a polymer-based membrane which ensures uniform dosing over a long period. The desired release rate can be controlled via the choice of polymer and via the thickness of the membrane.

Suitable polymers for the membrane are in principle the same polymers as those for the core (the central rod). Mention must be made here, for example, of polydimethylsiloxane which may optionally be fluorinated, or else mixtures of different polymers. Membrane thickness is preferably around 0.5 mm.

The membrane is applied by firstly swelling a tubing (membrane) prepared from the desired polymer in a solvent and then pressing the core containing the active ingredient into the still swollen tubing. The ends of the tubing are then preferably also sealed by a stopper, preferably consisting of the same material as the tubingmembrane, in order to counteract “bleeding” of the active ingredient at the ends of the tubing, which may result in a “burst effect” during use. The tubing may also be bonded with silicone in place of the stoppers.

Rods releasing a daily dose in the range of 1 500 μg of the particular active ingredient (A) or (B) may be employed according to the invention.

The release rate of active ingredient (A) may be chosen here to be half of that of active ingredient (B), owing to the higher efficacy of the former.

Thus, the resulting preferred dose range for the active ingredient (A) is 1-200 μg/day, with particular preference being given to the range of 1-100 μg and in particular the range of 2-50 μg/day. The preferred dose range of active ingredient (B) is 2-500 μg/day, with particular preference being given to the range of 2-200 μg and in particular the range of 5-100 μg/day.

The examples below serve to illustrate the invention.

The progestins employable according to the invention, 18-methyl-15β,16β-methylene-19-nor-20-spirox-4-en-3-one (compound A) or 18-methyl-6α,7α,15β,16β-dimethylene-19-nor-20-spirox-4-en-3-one (compound B), are prepared as described in WO 2008000521 (compound A: example 14 f; compound B: example 2).

The process of preparing the active ingredient-charged rods used in the rat experiment described below was carried out similarly to the process of preparing the active ingredient reservoirs, as described for an IUS usable in humans, for example (see for example EP 0 652 738 B1). Polymers which may be used for preparing the rod are polysiloxanes and modified polysiloxane polymers (see for example EP 0652738 BI, WO 0029464 and WO 0000550).

Specifically, first an active ingredient-charged core was prepared by vulcanizing a mixture of polyethylene oxide block-polydimethylsiloxane copolymer (PEO-b-PDMS), polydimetnylsiloxane and 10 per cent by weight of the active ingredient (in this case the particular progestin A or B), using a Pt (0)-divinyltetramethyldisiloxane catalyst.

It is also possible to use polydimethylsiloxane (PDMS) rather than PEO-b-PDMS, with bis(2,4-dichlorobenzoyl) peroxide having been used here as the vulcanization catalyst.

To prepare the active ingredient-containing core, a vertical piston unit with a corresponding nozzle head was used. The dimensions of the nozzle head were such that the outer diameter of the active ingredient-containing core is about 1 mm.

The active ingredient-containing core prepared in this way is then coated with a membrane consisting of PDMS, polytrifluoropropylmethylsiloxane (PTFPMS) or a PTFPMS/PDMS elastomeric mixture (75% PTFMPS, 25% PDMS). The inner diameter of the membrane material was ˜1 mm, with an outer diameter of ˜1.5 mm.

The coating was carried out by cutting the core and the membrane to a length of 10-15 mm, with the membrane being slightly longer (respectively approx. 1 mm at either end) than the core, in order to enable the ends of the membrane to be sealed with a small stopper after the core has been inserted. In order to enable the core to be inserted into the membrane, the latter was first made to swell in a cyclohexane or acetone-hexane mixture. The active ingredient-containing core was then pushed into the swollen membrane. Finally, the ends of the tubing were either bonded with silicone or sealed with a small stopper made of PTFPMS.

### EXAMPLE 1

The local uterine action of the progestin compared to systemic side effects (dissociation) was investigated on the basis of studies using rats. The uterus of ovary-resected rats responds to implantation of progestin-containing IUS (rods) with decidualization and weight gain. The local progestin effects were also determined on the basis of changes in gene expression.

Serum levels of luteinizing hormone (LH) are used for detecting systemic effects of the locally administered progestin. Basal serum-LH levels of ovary-resected rats are elevated compared to the LH levels of intact control animals. Undesired systemic efficacy of the uterine-administered progestin can be detected by a decrease in the LH level.

Method

Ovary-resected female rats were treated with estradiol (E2) for three days (0.2 μg/day/animal, subcutaneous dosing). On day 4, an IUS (rod) was implanted into the right uterine horn of each animal. The left uterine horn remained untreated for internal comparison. Administration of E2 was continued with a daily dose of 0.1 μg/animal to ensure responsiveness of the uterus (maintaining progesterone-receptor expression) to progestins. Blood was taken for LH level measurements on days 4, 10 and 17.

Performing the gene expression analyses:

The uterine tissue was homogenized in 800 μl of RLT lysis buffer (Qiagen, Hilden, Germany; #79216) using a Precellys24 homogenizer (Peqlab, Erlangen, Germany; 2.8 mm ceramic beads; #91-PCS-CK28, 2×6000 rpm). 400 μl of the homogenate obtained were used for isolating total RNA, using the QIAsymphony RNA kit (Qiagen, #931636) on a QIAsymphony SP robot for automated sample preparation. Reverse transcription of from 1 μg to 4 μg of total RNA was carried out using the SuperScript III first-strand synthesis system (Invitrogen, Carlsbad, USA; #18080-051) according to the random hexamer procedure.

Gene expression analysis was carried out with from 50 ng to 200 ng of cDNA per reaction on an SDS7900HT Real.time PCR system (Applied Biosystems, Carlsbad, USA) using TaqMan probes (Applied Biosystems; IGFBP-1 Rn00565713_m1, Cyp26a1 Rn00590308_m1, PPIA Rn00690933_m1) and the Fast Blue qPCR MasterMix Plus (Eurogentec, Liège, Belgium; #RT-QP2X-03+FB). For relative quantification, cyclophilin A (PPIA) was used as an endogenous control. Relative expression levels were calculated according to the comparative delta delta CT method.

Results

18-Methyl-15β,16β-methylene-19-nor-20-spirox-4-en-3-one (compound A) and 18-methyl-6α,7α,15β,16β-bis-methylene-19-nor-20-spirox-4-en-3-one (compound B) exhibited dose-dependent local efficacy by way of weight gain in the IUS-carrying uterine horn (FIG. 1/4).

Within the dose range tested (for compound A: 0.6-10 μg per animal and day, and for compound B: 1-45 μg/animal and day) both progestins surprisingly exhibited no LH decrease and therefore no systemic side effect, with the exception of the 10 μg/animal and day dose of compound A (FIG. 2/4).

The pharmacokinetic profile of 18-methyl-15β,16β-methylene-19-nor-20-spirox-4-en-3-one and 18-methyl-6α,7α,15β,16β-bis-methylene-19-nor-20-spirox-4-en-3-one, respectively, indicated a very fast break-down rate in all in-vitro metabolic studies (liver) as well as in all animal species studied in vivo.

With local administration by means of IUS (rods) in rats, compound A exhibited a 4- to 7-fold higher potency in inducing gene expression of relevant marker genes than levonorgestrel, with identical release rates (FIG. 3/4). This higher local potency additionally supports the possibility of achieving more rapid and stronger local gestagenic effects on the uterus without causing systemic side effects in the process.

As a result, these progestins can be dosed with local efficacy in such a way that the side effects described for levonorgestrel do not occur in the woman.

Very rapid break-down rates have also been found in vitro (liver) for humans. The rapid in-vitro breakdown in the liver may also indicate rapid in-vivo breakdown, resulting in a very low systemic exposition of 18-methyl-15β,16β-methylene-19-nor-20-spirox-4-en-3-one and 18-Methyl-6α,7α,15β,16β-dimethylene-19-nor-20-spirox-4-en-3-one after administration through an IUS being calculated. The expected substance levels (Css=concentration at steady state) are calculated from the rate of release from the IUS divided by the clearance. Using a dose of 20 μg per day and woman, which corresponds to that of Mirena, gives a calculated systemic exposition (load) for 18-methyl-15β,16β-methylene-19-nor-20-spirox-4-en-3-one and 18-methyl-6α,7α,15β,16β-dimethylene-19-nor-20-spirox-4-en-3-one, which is over 30 times lower in comparison with Mirena®.

### EXAMPLE 2

The action on the human androgen receptor was studied by means of transactivation analyses. For this, different concentrations of the test substances were to cells with stable expression of the human androgen receptor, and activation of the androgen receptor can be detected via a reporter gene.

Method

For transactivation studies, PC3 (human prostate carcinoma) cells which have been stably transfected with hAR and the MTV-luc reporter gene were used. The culture medium used was RPMI medium (without L-glutamine; without Phenol Red) #E15-49 PAA L-glutamine 200 mM #25030-024 Gibco BRL 100 U 100 μg/ml penicillinstreptomycin Gibco # 15140-122, with 10% foetal calf serum (FCS). The cells were cultured at 37° C. and 5% CO₂. The test medium corresponded to the culture medium, except that 10% FCS was replaced with 5% activated carbon-treated FCS (CCS). Cells were seeded in wells of a 96 well plate (“CulturPlate” from Packard #6005180) with 2×104 cells/well/200 μl of test medium. The cells were incubated with different concentrations of the test substances, and 80 μl of substrate were measured using the “steadylite HTS Reporter Gene Assay System” from Perkin Elmer.

Results

The results show that compound A (18-methyl-15β,16β-methylene-19-nor-20-spirox-4-en-3-one) and compound B (18-methyl-6α,7α,15β,16β-dimethylene-19-nor-20-spirox-4-en-3-one) have an EC50 in hAR transactivation which is more than 10 times higher than that of levonorgestrel: While the EC50 values are 6.9 nM for compound A and 56 nM for compound B, levonorgestrel has an EC50 of only 0.5 nM. This >10-fold dissociation over levonorgestrel means that no systemic androgenic effects are expected when the compounds are used, even if local uterine uses were to produce systemic active ingredient levels as those observed for levonorgestrel with Mirena® uses.

### EXAMPLE 3

The amounts of active ingredient (A) or (B) released were determined by means of reversed-phase liquid chromatography with UV detection in a 1% strength 2-hydroxypropyl-β-cyclodextrin (2-HPBCD) solution.

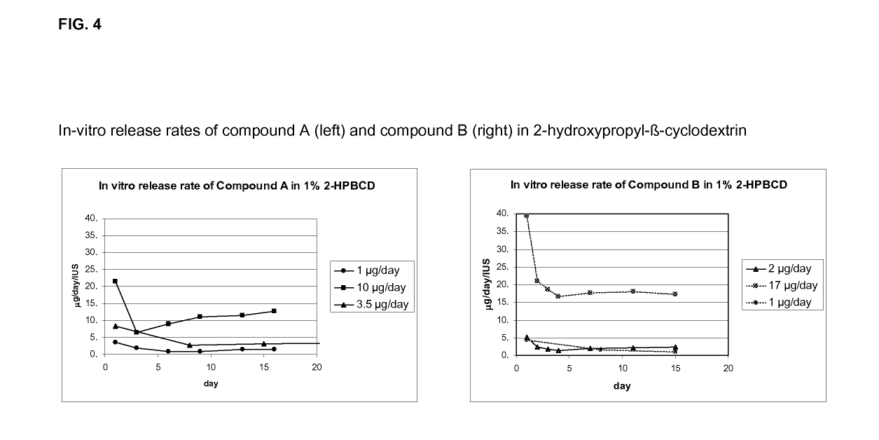

The in-vitro release rates stated in FIG. 4/4 were determined for a rod enveloped by a PTFPMS membrane.

1. A method for the treatment of uterine bleeding disorders or haemorrhages comprising the step of administering 18-Methyl-15β,16β-methylene-19-nor-20-spirox-4-en-3-ones of the general formula I, 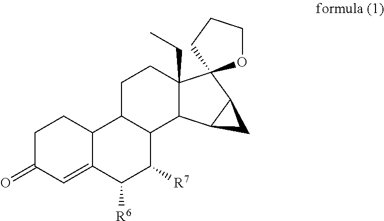
wherein R⁶ and R⁷ are a hydrogen atom or together are an a-methylene group, to a patient in need thereof.

2-3. (canceled)

4. The method according to claim 1, characterized in that 18-methyl-15β,16β-methylene-19-nor-20-spirox-4-en-3-one is administered by the intrauterine route.

5. (canceled)

6. The method according to claim 1, characterized in that 18-methyl-15β,16β-methylene-19-nor-20-spirox-4-en-3-one is administered by way of a daily dose of 1-200 μg.

7. The method according to claim 1, characterized in that 18-methyl-15β,16β-methylene-19-nor-20-spirox-4-en-3-one is administered by way of a daily dose of 1-100 μg.

8. The method according to claim 1, characterized in that 18-methyl-15β,16β-methylene-19-nor-20-spirox-4-en-3-one is administered by way of a daily dose of 2-50 μg.

9. The method according to claim 1, characterized in that 18-Methyl-15β,16β-methylene-19-nor-20-spirox-4-en-3-one is 18-methyl-6α,7α,15β,16β-dimethylene-19-nor-20-spirox-4-en-3-one.

10-14. (canceled)
